(19) United States
(12) Patent Application Publication
(10) Pub. No.: US 2021/0315724 A1
(43) Pub. Date: Oct. 14, 2021

(54) WAIST, HIP, AND PELVIS ADJUSTMENT BELT

(71) Applicant: Make Life International Enterprise Co., Ltd., Taichung (TW)

(72) Inventor: Yen-Ting CHIANG, Taichung (TW)

(21) Appl. No.: 16/945,529

(22) Filed: Jul. 31, 2020

(30) Foreign Application Priority Data
Apr. 9, 2020 (TW) 109111891

(51) Int. Cl. A61F 5/02; A61F 5/03

(52) U.S. Cl. CPC A61F 5/028; A61F 5/03

(57) ABSTRACT

A waist, hip and pelvis adjustment belt includes a waist, hip and pelvis adjustment belt assembly and plural ore bodies distributed and accommodated in the waist, hip and pelvis adjustment belt assembly. When a user wears the waist, hip and pelvis adjustment belt assembly to have the effect of lifting and binding the user's waist and hip, the ore bodies in the waist, hip and pelvis adjustment belt assembly will release far infrared light to generate a resonance frequency to promote blood circulation of the user's lower limbs, and restore and adjust the user's pelvis and hip joint, so that the user's piriformis and nerve will not be pressed to achieve the effect of good functionality and structural stability.

### FIELD OF THE INVENTION

The present invention relates to the technical field of waist, hip and pelvis adjustment belts, and more particularly to a waist, hip and pelvis adjustment belt applied for binding and restoring bones of the lower limbs and promoting blood circulation of the lower limbs.

### BACKGROUND OF THE INVENTION

With reference to FIG. 6 for the structure of a conventional restoration binding band, the binding band 30 is in an integral large-area form and comprises a covering face 31, and a male coupling portion 32 and a female coupling portion 33 respectively and extended outwardly from both sides of the covering face 31, and the covering face 31 is designed to cover a user's waist and buttock, and the male coupling portion 32 and the female coupling portion 33 are combined securely with each other, so that the covering face 31 binds and tightens the user's waist and buttock and lifts the user's buttock and binds and tightens the user's waist and hip bones, so as to promote blood circulations of the lower limbs. However, the binding band 30 is a covering face 31 in an integral large-area form, and the breathability and comfortability are poor, and the binding, tightening, and covering effects are low. Obviously, the conventional binding band requires improvements.

### SUMMARY OF THE INVENTION

In view of the poor functionality and structural stability of the conventional restoration binding band, the present invention provides a waist, hip and pelvis adjustment belt to overcome the drawbacks of the prior art.

To achieve the aforementioned and other objectives, the present invention discloses a waist, hip and pelvis adjustment belt comprising a waist, hip and pelvis adjustment belt assembly and a plurality of ore bodies, and the waist, hip and pelvis adjustment belt assembly comprises a waist binding strap body, an extending and connecting strap body, and a hip lifting and binding strap body. The waist binding strap body has a waist support tab disposed at the middle section of the waist binding strap body, and the waist support tab has an elastic stretching and fixing strap extended from both sides of the waist support tab separately, and a waist auxiliary binding strap extended from both sides of the middle section of the waist support tab separately. The two elastic stretching and fixing straps and the two waist auxiliary binding straps have a fixed binding portion separately, wherein the fixed binding portions are male and female buckles or male and female connectors a Velcro tape, and the waist support tab is hollow and has a plurality of accommodating spaces therein. The extending and connecting strap body has a top integrally coupled with the waist support tab of the waist binding strap body and a bottom integrally coupled with the hip binding belt body, and the hip binding belt body has an elastic lifting and fixing strap extended outwardly both sides of the hip binding belt body, and the two elastic lifting and fixing straps have a fixed binding portion separately, and the fixed binding portions are male and female buckles or male and female connectors of a Velcro tape, and the ore body is a Taichi stone, wherein the Taichi stone is formed by a silicon dioxide mineral with a purity of 70%-90%, and the silicon dioxide mineral contains trace elements such as diamond, nickel, manganese, copper, iron, and selenium, and a high-pressure molding machine is used to perform high-pressure molding by a pressure of 3 tons, and a high-temperature sintering device is used to produce the ore bodies with a high temperature of 1300 degrees, so that the ore body will release far infrared light to generate a resonance frequency. The ore bodies are distributed and accommodated in the accommodating space of the waist support tab disposed at the waist binding strap body of the waist, hip and pelvis adjustment belt assembly.

When a user wears the waist, hip and pelvis adjustment belt assembly, the waist support tab of the waist binding strap body is positioned at the user's waist, and the fixed binding portions of the elastic stretching and fixing strap and the waist auxiliary binding strap are bound and fixed to each other, and the hip binding belt body pulls and holds the user's buttock. After the elastic lifting and fixing strap is crossed and stretched, the fixed binding portion is fixed and combined with the waist binding strap body to provide the functions of lifting and binding the user's waist and buttock, and the ore bodies will release far infrared light from the accommodating space in the waist support tab of the waist binding strap body to generate a resonance frequency, so as to promote blood circulation of the user's lower limbs and prevent the user's piriformis and nerve from being pressed, and achieve the effect of good functionality and structural stability.

### BRIEF DESCRIPTION OF THE DRAWINGS

FIG. 1 is a schematic view showing a front unfolded status of a waist, hip and pelvis adjustment belt of the present invention;

FIG. 2 is a schematic view showing a back unfolded status of a waist, hip and pelvis adjustment belt of the present invention;

FIG. 3 is a partial side cross-sectional view of a waist, hip and pelvis adjustment belt of the present invention;

FIG. 4 is a schematic view showing an application of a waist, hip and pelvis adjustment belt worn by a user in accordance with the present invention;

FIG. 5 is a schematic view showing a user wearing a waist, hip and pelvis adjustment belt while performing a squatting action in accordance with the present invention;

FIG. 6 is a schematic view of the structure of a conventional restoration binding band of a waist, hip and pelvis adjustment belt of the present invention; and

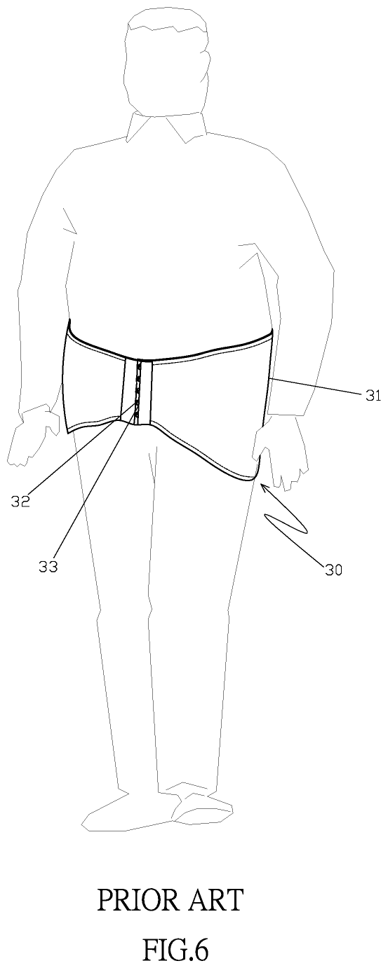

FIG. 7 is a far infrared emissivity waveform chart of a far infrared release test report provided by the Industrial Technology Research Institute of Taiwan.

### DESCRIPTION OF THE PREFERRED EMBODIMENTS

The objectives, technical characteristics and effects of the present invention will become apparent with the detailed description of preferred embodiments accompanied with the illustration of related drawings. It is intended that the embodiments and drawings disclosed herein are to be considered illustrative rather than restrictive.

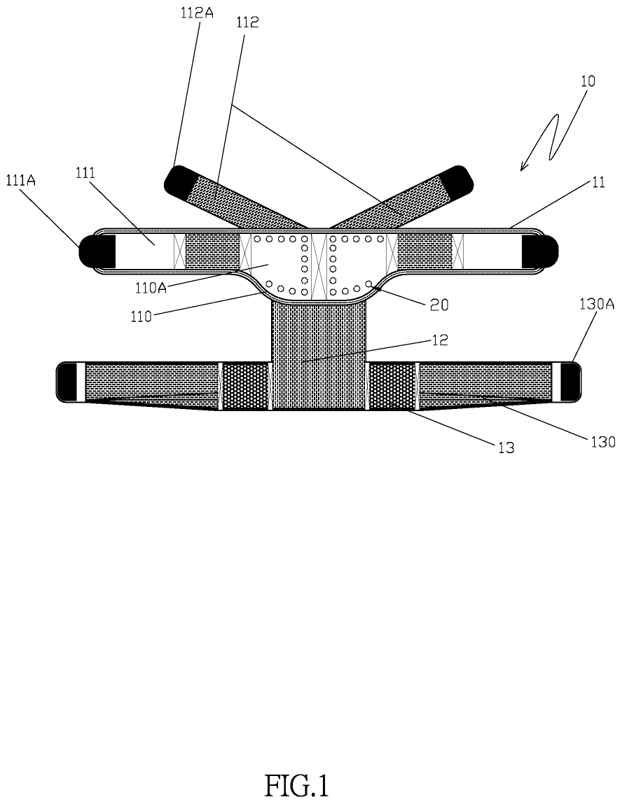

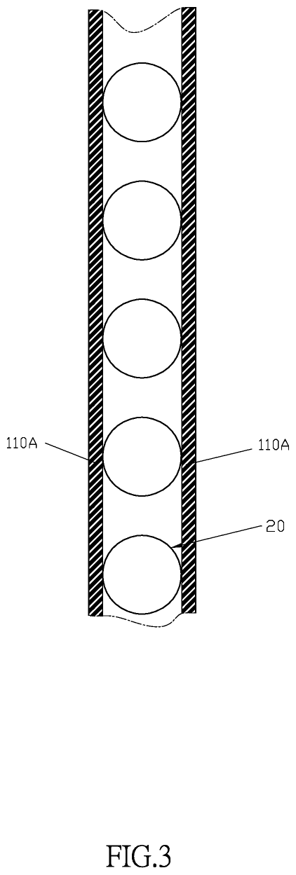

With reference to FIGS. 1 to 3 for the schematic views showing the front unfolded status and the back unfolded status of a waist, hip and pelvis adjustment belt and the partial side cross-sectional view of the waist, hip and pelvis adjustment belt in accordance with the present invention respectively, the waist, hip and pelvis adjustment belt comprises:

a waist, hip and pelvis adjustment belt assembly 10, having a waist binding strap body 11, an extending and connecting strap body 12 and a hip lifting and binding strap body 13, and having a waist support tab 110 disposed at the middle section of the waist binding strap body 11, an elastic stretching and fixing strap 111 extended outwardly from both sides of the waist support tab 110, and a waist auxiliary binding strap 112 extended separately from both sides of the middle section of the waist support tab 110, wherein the two elastic stretching and fixing straps 111 and the two waist auxiliary binding straps 112 have fixed binding portions 111A, 112A respectively, and the fixed binding portions 111A, 112A are male and female buckles or male and female connectors of a Velcro tape, and the waist support tab 110 is hollow and has a plurality of accommodating spaces 110A therein, and the extending and connecting strap body 12 has a top integrally coupled with the waist support tab 110 of the waist binding strap body 11 and a bottom integrally coupled with the hip binding belt body 13, and an elastic lifting and fixing strap 130 is extended outwardly from both sides of the hip binding belt body 13, and the two elastic lifting and fixing straps 130 have a fixed binding portion 130A separately, and the fixed binding portions 130A are male and female buckles or male and female connectors of a Velcro tape; and

a plurality of ore bodies 20, which are Taichi stones formed by a silicon dioxide mineral with a purity of 70%˜90%, and the silicon dioxide mineral contains trace elements such as diamond, nickel, manganese, copper, iron, and selenium, and a high-pressure molding machine is used to perform high-pressure molding by a pressure of 3 tons, and a high-temperature sintering device is used to produce the ore bodies 20 with a high temperature of 1300 degrees, so that the ore body 20 will release far infrared light to generate a resonance frequency.

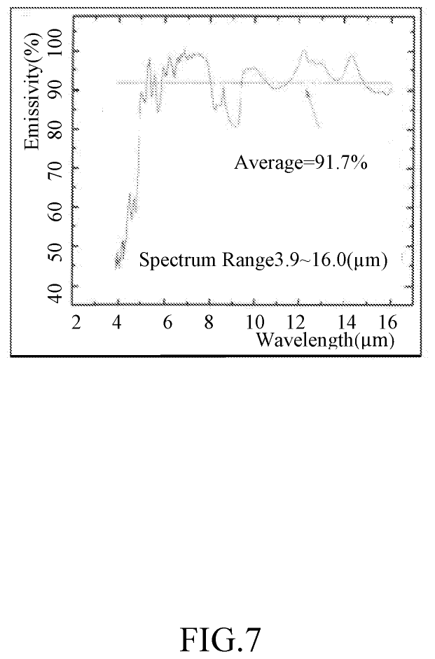

FIG. 7 shows the far infrared emissivity waveform chart of a far infrared release test report by the Industrial Technology Research Institute of Taiwan, and Table 1 shows the far infrared light test report by the Far Infrared Association (Japan),

TABLE 1
Average Emissivity

| Name of Testing | | |
|---|---|---|
| Material | \textcircled{1} | \textcircled{2} |
| Mount Everest's | 93.32 | 93.36 |
| Alpha Stone | | |
| (Taichi Stone) | | |

(Unit in %)
The average emissivity is calculated by the following method:
Wavelength Range \textcircled{1} 4~15 microns; \textcircled{2} 6~14 microns, and the numerical values of the emissivity are used to calculate the average.

In addition, the release of far infrared light improves the blood flow and the blood flow speed. Without the use of Taichi stone, the blood flow is 50.6 (100%) and the blood flow rate is 3.9 (100%). With the use of Taichi stone, the blood flow is increased to 53.3 (105%) and the blood flow speed is increased to 4.5 (115%). Specific details are given in the blood flow and blood flow speed test report by the Far Infrared Association (Japan) as shown in Table 2 below.

TABLE 2

| Rate of Change | |
|---|---|
| | Mount Everest's |
| | Alpha Stone |
| Normal | (Taichi Stone) |

| | | | | |
|---|---|---|---|---|
| Blood Flow | 50.6 | 100% | 53.3 | 105% |
| Blood Flow | 3.9 | 100% | 4.5 | 115% |
| Speed | | | | |

The data above show that the Taichi stone has the effect of releasing far infrared light energy continuously, and the ore bodies 20 are distributed and accommodated in the accommodating space 110A of the waist support tab 110 disposed at the waist binding strap body 11 of the waist, hip and pelvis adjustment belt assembly 10.

With the aforementioned structure, the waist, hip and pelvis adjustment belt of the present invention is completed.

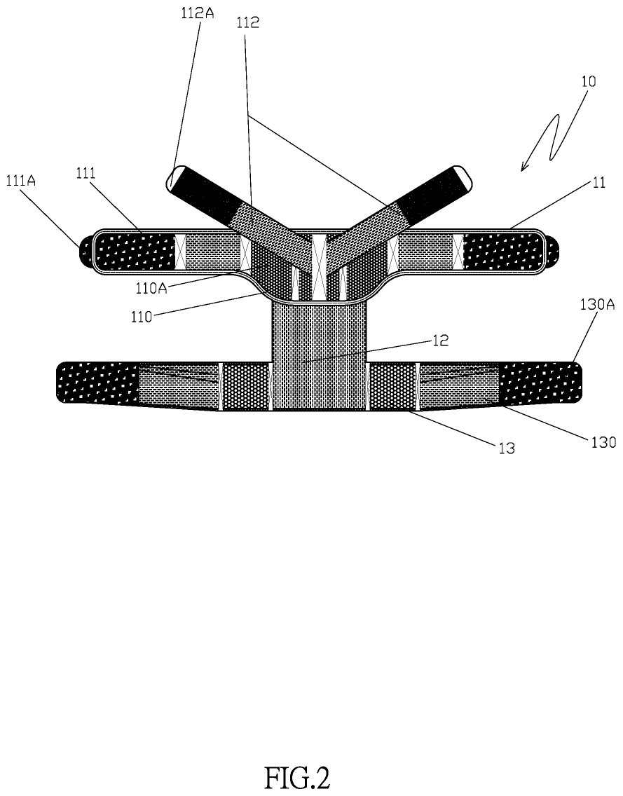

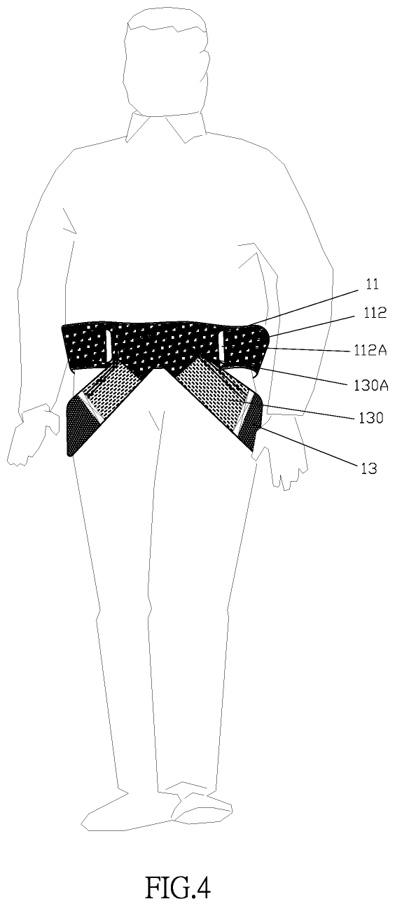

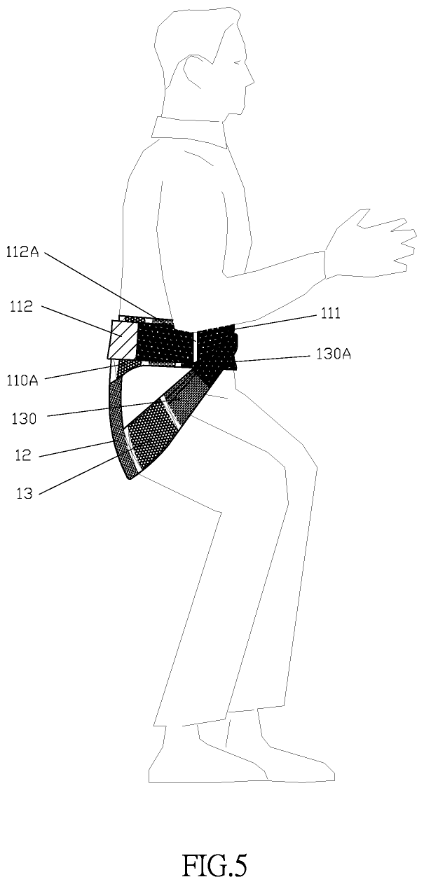

With reference to FIGS. 2 to 5 for the schematic view showing the back unfolded status of a waist, hip and pelvis adjustment belt, the partial side cross-sectional view of the waist, hip and pelvis adjustment belt, the schematic view of wearing the waist, hip and pelvis adjustment belt, and the schematic view of a user wearing the waist, hip and pelvis adjustment belt while performing a squatting action in accordance with the present invention respectively, and the ore bodies 20 are distributed and accommodated in the accommodating space 110A of the waist support tab 110 disposed at the waist binding strap body 11 of the waist, hip and pelvis adjustment belt assembly 10.

When a user wears the waist, hip and pelvis adjustment belt assembly 10, the waist support tab 110 of the waist binding strap body 11 is positioned at the user's waist, and the fixed binding portions 111A, 112A of the elastic stretching and fixing strap 111 and the waist auxiliary binding strap 112 are bound and fixed, and the hip binding belt body 13 pulls and holds the user's buttock, and after the elastic lifting and fixing strap 130 is crossed and stretched, the fixed binding portion 130A is fixed and combined with the waist binding strap body 11 to provide the functions of lifting and binding the user's waist and buttock, and the ore bodies 20 will release far infrared light from the accommodating space 110A in the waist support tab 110 of the waist binding strap body 11 to generate a resonance frequency, so as to promote blood circulation of the user's lower limbs and prevent the user's piriformis and nerve from being pressed, and achieve the effect of good functionality and structural stability.

In summation of the description above, the present invention can surely achieve the aforementioned functions and objectives and comply with the patent application requirements, and thus is duly filed for patent application.

What is claimed is:

1. A waist, hip and pelvis adjustment belt, comprising:
a waist, hip and pelvis adjustment belt assembly, having a waist binding strap body, an extending and connecting strap body, and a hip lifting and binding strap body, and the waist binding strap body having a waist support tab disposed at the middle section thereof, an elastic stretching and fixing strap extended outwardly from both sides of the waist support tab, and the two elastic stretching and fixing straps having a fixed binding portion, and the waist support tab including a plurality of accommodating spaces, and the extending and connecting strap body having a top integrally coupled with the waist support tab of the waist binding strap body, and a bottom integrally coupled with the hip binding belt body, and the hip binding belt body having an elastic lifting and fixing strap extended outwardly from both sides thereof, and the two elastic lifting and fixing straps having a fixed binding portion; and
a plurality of ore bodies, which are Taichi stones, distributed and accommodated in an accommodating spaces of the waist support tab disposed at the waist binding strap body of the waist, hip and pelvis adjustment belt assembly.

2. The waist, hip and pelvis adjustment belt as claimed in claim 1, wherein the waist support tab has a waist auxiliary binding strap extended from both sides of the middle section of the waist support tab, and the two waist auxiliary binding straps have a fixed binding portion separately.

3. The waist, hip and pelvis adjustment belt as claimed in claim 2, wherein the waist auxiliary binding strap of the waist binding strap body has a fixed binding portion, and the fixed binding portions are bound and fixed with each other.

4. The waist, hip and pelvis adjustment belt as claimed in claim 2, wherein the fixed binding portion are male and female buckles or male and female connectors of a Velcro tape respectively.

5. The waist, hip and pelvis adjustment belt as claimed in claim 1, wherein the fixed binding portions of the elastic lifting and fixing strap are male and female buckles or male and female connectors of a Velcro tape respectively.

6. The waist, hip and pelvis adjustment belt as claimed in claim 1, wherein the Taichi stone is formed by a silicon dioxide mineral with a purity of 70%˜90%, and the silicon dioxide mineral contains trace elements selected from the group of diamond, nickel, manganese, copper, iron, and selenium, and a high-pressure molding machine is used to perform high-pressure molding by a pressure of 3 tons, and a high-temperature sintering device is used to produce the ore bodies with a high temperature of 1300 degrees, so that the ore body will release far infrared light to generate a resonance frequency.

7. The waist, hip and pelvis adjustment belt as claimed in claim 1, wherein the fixed binding portions of the elastic stretching and fixing strap of the waist binding strap body are bound and fixed to each other, and the elastic lifting and fixing straps of the hip binding belt body are crossed and stretched and then fixed and combined to the waist binding strap body by the fixed binding portion.
